(19) United States
(12) Patent Application Publication
(10) Pub. No.: US 2014/0364953 A1
(43) Pub. Date: Dec. 11, 2014

(54) UNIQUE BASEPLATE FOR REVERSE AND TOTAL SHOULDER REPLACEMENT WITH ADDED FIXATION TO THE SCAPULA

(71) Applicants: Daniel P. Tomlinson, Monroe, NY (US); Rocco Bassora, Central Valley, NY (US)

(72) Inventors: Daniel P. Tomlinson, Monroe, NY (US); Rocco Bassora, Central Valley, NY (US)

(21) Appl. No.: 13/912,315

(22) Filed: Jun. 7, 2013

(51) Int. Cl. A61F 2/40

(52) U.S. Cl. CPC A61F 2/4081
USPC 623/19.11

(57) ABSTRACT

A unique baseplate for reverse and total shoulder replacement with added fixation to the scapula is disclosed. It adds increased fixation to the already existing fixation devices on the market to allow the indications for reverse shoulder replacement surgery to be expanded. It also allows for a standard shoulder replacement to be performed with intact rotator cuff and poor glenoid bone stock.

### REFERENCE TO RELATED APPLICATION

This application claims the benefit of the filing date of U.S. Provisional Application Ser. No. 61/659,849, entitled “A unique baseplate for reverse and total shoulder replacement with added fixation to the scapula” and filed on Jun. 14, 2012. The teachings of the entire referenced application are incorporated herein by reference.

### FIELD OF THE INVENTION

This invention relates generally to a stable reverse and standard total shoulder replacement in patients with poor glenoid bone stock or bone loss. More specifically, the invention relates to obtaining stable and adequate glenoid fixation, especially for a glenoid baseplate for fixation into the base of the coracoid or superior aspect of the glenoid.

### BACKGROUND OF THE INVENTION

Obtaining stable and adequate glenoid fixation is very necessary to do a stable reverse and standard total shoulder replacement in patients with poor glenoid bone stock or bone loss.

The current glenoid baseplates must be seated on the inferior aspect of the glenoid and do not allow for fixation into the base of the coracoid or superior aspect of the glenoid. The current glenoid polyethylene component for primary total shoulder cannot be fixated adequately to a glenoid with significant bone loss and does not allow for a primary total shoulder in a single surgery for these types of patients.

There is a demand to add increased fixation to the already existing fixation devices on the market to allow the indications for reverse shoulder replacement surgery to be expanded. It also allows for a standard shoulder replacement to be performed with intact rotator cuff and poor glenoid bone stock. There are no implants on the market that allows for this situation.

### BRIEF DESCRIPTION OF THE DRAWINGS

FIG. 1 is a perspective view of a glenoid baseplate for fixation into the base of the coracoid or superior aspect of the glenoid.

FIG. 2 is a perspective view of a glenoid baseplate with polyethylene components for fixation into the base of the coracoid or superior aspect of the glenoid.

FIG. 3 is a perspective view of polyethylene glenoid.

### DETAILED DESCRIPTION OF THE PREFERRED EMBODIMENTS

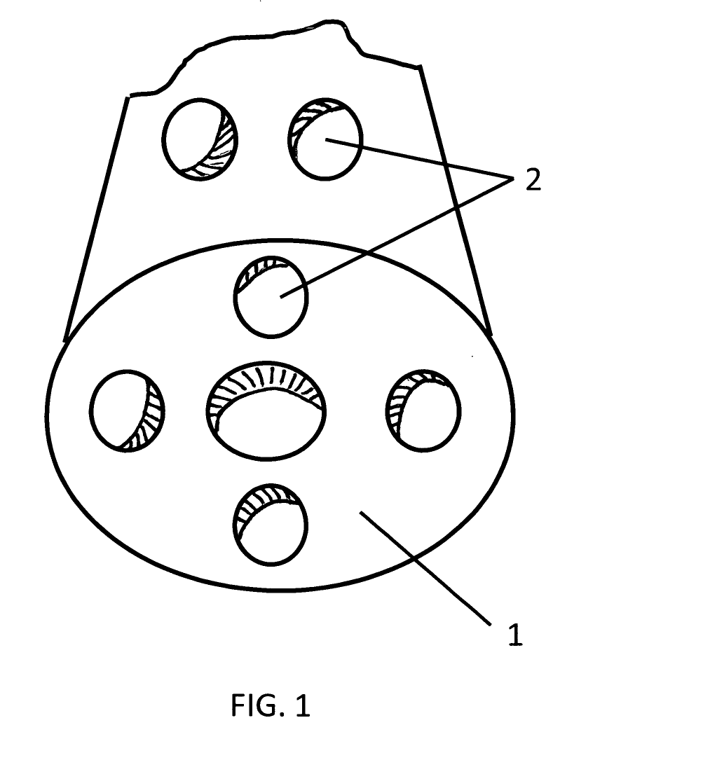

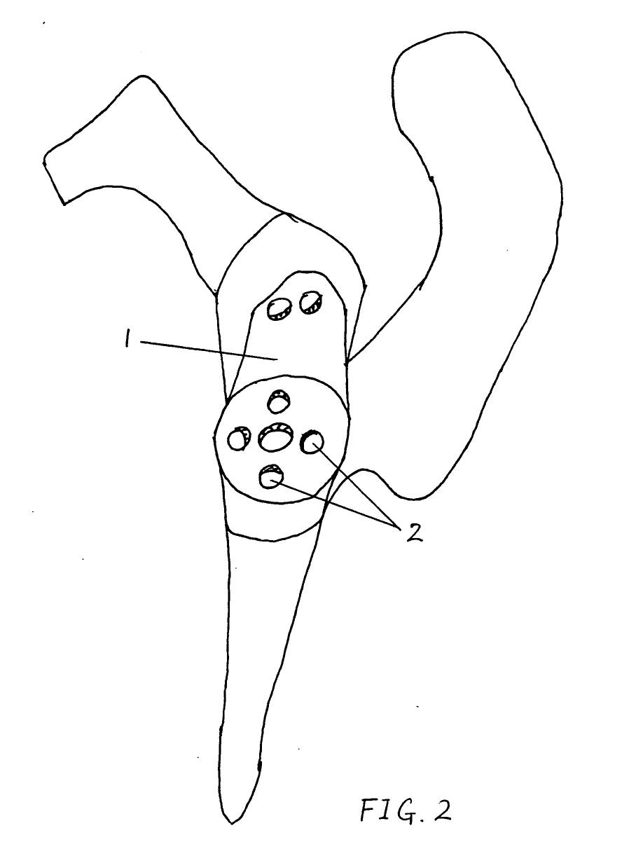

As stated above, obtaining stable and adequate glenoid fixation is necessary to do a stable reverse and standard total shoulder replacement in patients with poor glenoid bone stock or bone loss. The present invention, a glenoid baseplate, claimed here solves this problem. Such glenoid baseplate has a superior extension with additional screw hole(s) that can be directed into the coracoid base and superior scapula. The baseplate can accept a glenosphere or a standard polyethylene component for reverse or standard total shoulder replacement depending on the integrity of the rotator cuff. It still has the standard screw holes available for addition (See FIG. 1 and FIG. 2).

The claimed invention differs from what currently exists. The present invention is a significant improvement on what currently exists since there is no baseplate that allows for added fixation into the base of coracoid or superior scapula and there is also no implant that allows for primary glenoid implantation in patients with intact rotator cuffs and poor quality glenoid bone stock.

In patients with poor bone quality or glenoid bone loss, especially in revision surgery, the baseplate fixation will be pull out of the weak bone and the implant will fail. However, this invented glenoid baseplate adds increased fixation to the already existing fixation devices on the market to allow the indications for reverse shoulder replacement surgery to be expanded. It also allows for a standard shoulder replacement to be performed with intact rotator cuff and poor glenoid bone stock. There are no implants on the market that allows for this.

### The Components of the Invention Presented Here Include:

1. Baseplate (1) with superior extension—metal with in-growth or on-growth material on posterior surface, one or multiple locking or non-locking screw holes (2);

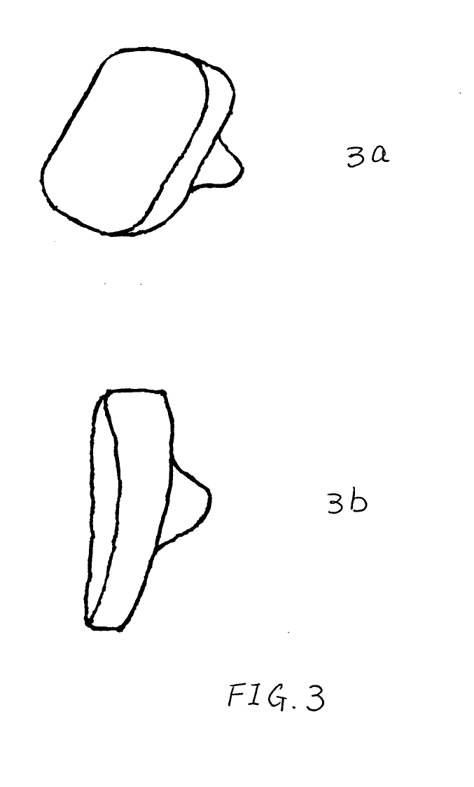

2. Polyethylene glenoid (3a, 3b) in FIG. 3 with metal back containing Morse Taper or possibly screw on mechanism.

Relationship between the Components:

Item 1, baseplate (1), is press fit into the glenoid and fixated with screws through the locking and/or non-locking screw holes. Item 2, polyethylene component (3a, 3b), fits via Morse Taper into the center of the baseplate.

### How the Invention Works:

Item 1, baseplate, is press fit to the glenoid after appropriate reaming. Appropriate screws are then placed into the appropriate holes for fixation. This can then accept a standard glenosphere via a Morse Taper, or possibly threaded screw in mechanism, for a reverse total shoulder. Or it can accept item 2, polyethylene component, via Morse Taper, or possibly threaded screw in mechanism, for a standard total shoulder replacement.

### How to Make the Invention:

The baseplate and polyethylene components would be made by an orthopedic manufacturer. There is a hole for a central screw. There are two to four peripheral screws around the circular or oval part of the plate. There are extra holes machined into the superior extension of the baseplate. Both standard “off the shelf” plates as well as customizable plates are available.

All elements are essential. More screw holes could be added to the baseplate to allow for more fixations.

The baseplate could be made with various shape extensions. It could have additional screw holes for additional extension. It could have more metal on any given side to correct for version of the glenoid. The screw holes could come in multiple angles with locking or non-locking features.

### How to Use the Invention:

A reamer is used to smooth the glenoid bone. The base plate is then impacted into the glenoid. The peripheral screws are first drilled and measured and the appropriate screws are inserted. The same technique is used for a larger central screw hole and the holes in the superior extension. The glenoid component can then be implanted with the Morse Taper into the central hole of the baseplate, or via a threaded screw in mechanism.

Disclosed here is a glenoid base plate specially designed for a stable reverse and standard total shoulder replacement in patients with poor glenoid bone stock or bone loss.

In summary, the present invention provides a novel, useful, and strong glenoid base plate, which is inexpensive and easy to manufacture. Such glenoid baseplate is very attractive to doctors who will do the shoulder replacements or related surgeries.

While particular embodiments of the invention have been shown in the drawings and described above, it will be apparent, that many changes may be made in the form, arrangement and positioning of the various elements of the combination. In consideration thereof it should be understood that preferred embodiments of this invention is only for illustration purpose and not intended to limit the scope of the invention. Although the glenoid baseplate and the method of using the same according to the present invention has been described in the foregoing specification with considerable details, it is to be understood that modifications may be made to the invention which do not exceed the scope of the appended claims and modified forms of the present invention done by others skilled in the art to which the invention pertains will be considered infringements of this invention when those modified forms fall within the claimed scope of this invention.

What is claimed is:

1. A glenoid baseplate used in shoulder replacements or related surgeries has a superior extension capability by having additional standard screw hole(s) in such baseplate, wherein said glenoid baseplate can be directed into a coracoid base or a superior scapula, wherein said glenoid baseplate can also accept a glenosphere or a standard polyethylene component for reverse or standard total shoulder replacement via Morse Taper or threaded screw in mechanism, wherein said baseplate is made with various shape for extensions.

2. The said glenoid baseplate in claim 1 has one or multiple locking or non-locking standard screw holes, wherein there is a screw hole in the central area of said baseplate, wherein there are two to four peripheral screw holes around the circular or oval part of said baseplate, wherein said screw holes designed with multiple angles and have locking and/or non-locking features, wherein there are extra screw holes built into the said baseplate for additional extension.

3. The said polyethylene component in claim 1 fits into the center of said baseplate via Morse Taper or threaded screw in mechanism in the central area of said baseplate.

4. A method to use said glenoid baseplate in claim 1 comprising the following steps:
(a) Use a reamer to smooth a glenoid bone;
(b) Press said glenoid baseplate into a glenoid after appropriate reaming and fix with screws through the locking and/or non-locking screw holes;
(c) Insert a standard glenosphere via a Morse Taper or threaded screw in mechanism for a reverse total shoulder replacement or insert a polyethylene component via a Morse Taper or threaded screw in mechanism for a standard total shoulder replacement.
